(19) United States
(12) Patent Application Publication
(10) Pub. No.: US 2021/0388322 A1
(43) Pub. Date: Dec. 16, 2021

(54) METHOD FOR PRODUCING INDUCED PLURIPOTENT STEM CELLS USING RNA NANOPARTICLES FOR CELL TRANSFORMATION

(71) Applicant: UNIVERSITY OF SEOUL INDUSTRY COOPERATION FOUNDATION, Seoul (KR)

(72) Inventors: Jong-Bum LEE, Seoul (KR); Hye-Jin KIM, Seoul (KR); Ju-Hyun PARK, Chuncheon-si (KR)

(21) Appl. No.: 17/288,470

(22) PCT Filed: Oct. 29, 2018
(86) PCT No.: PCT/KR2018/012933

§ 371 Date: May 20, 2021

(30) Foreign Application Priority Data
Oct. 26, 2018 (KR) 10-2018-0128832

(51) Int. Cl. C12N 5/074; C12N 15/87; C12N 15/113

(52) U.S. Cl. CPC C12N 5/0696; C12N 15/87; C12N 2310/141; C12N 2510/00; C12N 2310/14; C12N 15/113

(57) ABSTRACT

The present invention pertains to a method for producing induced pluripotent stem cells, and more specifically, to a method for producing induced pluripotent stem cells using RNA nanoparticles for cell transformation, wherein: cell transformation can be effectively performed without genetic modification by producing induced pluripotent stem cells using self-assembled RNA nanoparticles including at least one RNA selected from the group consisting of messenger RNA for expressing transcription factors which induce somatic cells and adult stem cells to be dedifferentiated into induced pluripotent stem cells, micro RNA facilitating the dedifferentiation process, and small interfering RNA; the production efficiency of iPSCs can be maximized by adjusting structural properties and activity; and low gene loading efficiency can be overcome by applying an infinite replication process to incorporate high concentrations of RNA in RNA nanoparticles.

### CROSS-REFERENCE TO RELATED APPLICATIONS

This application claims the benefit under 35 U.S.C. section 371, of PCT International Application No.: PCT/KR2018/012933, filed on Oct. 29, 2018, which claims foreign priority to Korean Patent Application No.: KR10-2018-0128832, filed on Oct. 26, 2018, in the Korean Intellectual Property Office, both of which are hereby incorporated by reference in their entireties.

### TECHNICAL FIELD

The present invention pertains to a method for producing induced pluripotent stem cells and, more specifically, to a method for producing induced pluripotent stem cells using RNA nanoparticles for cell transformation, wherein: cell transformation can be effectively performed without genetic modification by producing induced pluripotent stem cells using self-assembled RNA nanoparticles including at least one RNA selected from the group consisting of messenger RNA for expressing transcription factors which induce somatic cells and adult stem cells to be dedifferentiated into induced pluripotent stem cells, micro RNA facilitating the dedifferentiation process, and small interfering RNA; the production efficiency of iPSCs can be maximized by adjusting structural properties and activity; and low gene loading efficiency can be overcome by applying an infinite replication process to incorporate high concentrations of RNA in RNA nanoparticles.

### BACKGROUND ART

The induced pluripotent stem cells (iPSCs) are pluripotent stem cells obtained by artificially expressing specific genes to artificially induce pluripotency in adult somatic cells, which are non-pluripotent cells, and dedifferentiation of human somatic cells or adult stem cells into iPSCs is generally performed by the transfer of genes including four types of transcription factors (Oct4, Sox2, cMyc, and Klf4). For example, transferring genes including transcription factors to somatic cells can be performed by transferring the genes to somatic cells through a virus, as described in the following patent document.

### Patent Document

International Patent Publication No. WO 2013177133 (published on May 21, 2012) “Generation of human iPS cells by a synthetic self-replicative RNA”

However, in the method for transferring transcription factor genes to somatic cells using a virus, viral genes are inserted into the chromosomes of target cells, and thus unexpected genomic variations are caused, which increases the risk of developing cancer when iPSCs are clinically applied, and the produced iPSCs have a problem in that a process of differentiation into cells of a desired lineage is difficult to be controlled, compared to embryonic stem cells. In order to solve the aforementioned problems, a method for transferring episomal plasmid DNA or minicircle DNA, a method using Sendai virus which is a chromosomally unintegrated virus, a method for directly transferring messenger RNA or a protein, and the like can be considered, but the aforementioned methods also cannot exclude the chromosome insertion problem, or the methods cause a problem such as low production efficiency of iPSCs or causing cytotoxicity.

### DISCLOSURE

### Technical Problem

The present invention has been devised to solve the aforementioned problems, and

an objective of the present invention is to provide a method for producing induced pluripotent stem cells using RNA nanoparticles for cell transformation, in which cell transformation can be effectively performed without genetic modification by producing induced pluripotent stem cells using RNA nanoparticles including at least one RNA selected from the group consisting of messenger RNA for expressing transcription factors which induce somatic cells and adult stem cells to be dedifferentiated into induced pluripotent stem cells, micro RNA facilitating the dedifferentiation process, and small interfering RNA.

Moreover, another object of the present invention is to provide a method for producing induced pluripotent stem cells using RNA nanoparticles for cell transformation, in which the production efficiency of iPSCs can be maximized by adjusting structural properties (having the form of nanoparticles) and activity (RNA having various functions is included at the same time).

Furthermore, still another object of the present invention is to provide a method for producing induced pluripotent stem cells using RNA nanoparticles for cell transformation, in which low gene loading efficiency can be overcome by applying an infinite replication process to incorporate high concentrations of RNA in RNA nanoparticles.

### Technical Solution

In order to achieve the aforementioned objects, the present invention is implemented by embodiments having the following configuration.

According to one embodiment of the present invention, a method for producing induced pluripotent stem cells according to the present invention includes: a transfer step of transferring, to somatic cells or adult stem cells, RNA nanoparticles for cell transformation, which allow the somatic cells or the adult stem cells to be dedifferentiated into induced pluripotent stem cells, or facilitate the dedifferentiation; and a culture step of culturing the cells to which the RNA nanoparticles for cell transformation have been transferred after the transfer step to produce induced pluripotent stem cells.

According to another embodiment of the present invention, in the method for producing induced pluripotent stem cells according to the present invention, the RNA nanoparticles for cell transformation include at least one RNA selected from the group consisting of messenger RNA for expressing transcription factors which allow somatic cells or adult stem cells to be dedifferentiated into induced pluripotent stem cells, micro RNA facilitating the dedifferentiation, and small interfering RNA.

According to still another embodiment of the present invention, in the method for producing induced pluripotent stem cells according to the present invention, at least one RNA nanoparticle selected from the group consisting of messenger RNA nanoparticles for expressing transcription factors which allow somatic cells or adult stem cells to be dedifferentiated into induced pluripotent stem cells, micro RNA nanoparticles facilitating the dedifferentiation, small interfering RNA nanoparticles facilitating the dedifferentiation, complex RNA nanoparticles which include micro RNA facilitating the dedifferentiation and small interfering RNA facilitating the dedifferentiation, complex RNA nanoparticles which include messenger RNA for expressing a transcription factor and micro RNA facilitating the dedifferentiation, complex RNA nanoparticles which include messenger RNA for expressing a transcription factor and small interfering RNA facilitating the dedifferentiation, and complex RNA nanoparticles which include messenger RNA for expressing a transcription factor, micro RNA facilitating the dedifferentiation, and small interfering RNA facilitating the dedifferentiation is used as the RNA nanoparticles for cell transformation.

According to still another embodiment of the present invention, in the method for producing induced pluripotent stem cells according to the present invention, the RNA nanoparticles for cell transformation each have a spherical shape and have a diameter of 50 to 200 nm.

According to still another embodiment of the present invention, in the method for producing induced pluripotent stem cells according to the present invention, the RNA nanoparticles for cell transformation include messenger RNA nanoparticles for expressing transcription factors which allow somatic cells or adult stem cells to be dedifferentiated into induced pluripotent stem cells, complex RNA nanoparticles which include micro RNA facilitating the dedifferentiation and small interfering RNA facilitating the dedifferentiation, and complex RNA nanoparticles which include messenger RNA for expressing a transcription factor and micro RNA facilitating the dedifferentiation.

According to still another embodiment of the present invention, in the method for producing induced pluripotent stem cells according to the present invention, the messenger RNA nanoparticles are produced through a pDNA production step of producing plasmid DNA containing a base sequence complementary to a repeating messenger RNA base sequence for expressing transcription factors which allow somatic cells or adult stem cells to be dedifferentiated into induced pluripotent stem cells; and a particle formation step of incubating a reaction solution containing the plasmid DNA and an RNA polymerase at a certain temperature for a certain time, performing rolling circle transcription of the plasmid DNA using the RNA polymerase to produce long single-stranded messenger RNA containing the repeating messenger RNA base sequence for expressing transcription factors, and allowing the produced single-stranded messenger RNA to be self-assembled while being twisted and entangled, to thereby form nanoparticles.

According to still another embodiment of the present invention, in the method for producing induced pluripotent stem cells according to the present invention, the complex RNA nanoparticles, which include micro RNA facilitating the dedifferentiation and small interfering RNA facilitating the dedifferentiation, are produced through a first circular DNA production step of producing first circular DNA by complementarily binding a promoter to ssDNA which is for facilitating the dedifferentiation of somatic cells or adult stem cells into induced pluripotent stem cells, and includes a base sequence complementary to a micro RNA base sequence and a si complementary base sequence that is a base sequence complementary to a small interfering RNA base sequence; a second circular DNA production step of producing second circular DNA by complementarily binding a promoter to ssDNA containing a base sequence complementary to a micro RNA base sequence and a base sequence complementary to the si complementary base sequence; and a particle formation step of incubating a reaction solution containing the first circular DNA, the second circular DNA, and an RNA polymerase at a certain temperature for a certain time, performing rolling circle transcription of each of the first circular DNA and the second circular DNA using the RNA polymerase to form long single-stranded first RNA containing a repeating micro RNA base sequence and a small interfering RNA base sequence, and long single-stranded second RNA containing a repeating micro RNA base sequence and the base sequence complementary to the small interfering RNA base sequence, and partially complementarily binding the first RNA to the second RNA to be self-assembled while being entangled, to thereby form nanoparticles.

According to still another embodiment of the present invention, in the method for producing induced pluripotent stem cells according to the present invention, the complex RNA nanoparticles, which include messenger RNA for expressing a transcription factor and micro RNA facilitating the dedifferentiation, are produced through a pDNA production step of producing plasmid DNA containing a base sequence complementary to a messenger RNA base sequence for expressing a transcription factor and a first binding base sequence that is a base sequence that enables complementary binding to micro RNA; a circular DNA production step of producing circular DNA containing a base sequence complementary to a micro RNA base sequence and a base sequence complementary to the first binding base sequence; and a particle formation step of incubating a reaction solution containing the plasmid DNA, the circular DNA, and an RNA polymerase at a certain temperature for a certain time, performing rolling circle transcription of each of the plasmid DNA and the circular DNA using the RNA polymerase to form long single-stranded first RNA containing a repeating messenger RNA base sequence and the first binding base sequence, and long single-stranded second RNA containing a repeating micro RNA base sequence and a second binding base sequence, and partially complementarily binding the first RNA to the second RNA to be self-assembled while being entangled, to thereby form nanoparticles.

According to still another embodiment of the present invention, in the method for producing induced pluripotent stem cells according to the present invention, the method for producing induced pluripotent stem cells further includes a loading step of loading, on the RNA nanoparticles, a protein that inhibits an innate immune response, before the transfer step.

According to still another embodiment of the present invention, in the method for producing induced pluripotent stem cells according to the present invention, in the transfer step, an electroporation method or a method using a positively charged polymer is used, the electroporation method is performed by suspending somatic cells or adult stem cells in a resuspension buffer, adding RNA nanoparticles, and then applying an electric shock, and the method using a positively charged polymer is performed by dispensing somatic cells or adult stem cells into a culture dish, adding a growth medium, culturing the cells for a certain time, and then adding, to the growth medium, a complex formed by mixing RNA nanoparticles with a positively charged polymer.

### Advantageous Effects

The present invention can achieve the following effects by the aforementioned embodiments.

The present invention has the effect that cell transformation can be effectively performed without genetic modification by producing induced pluripotent stem cells using RNA nanoparticles including at least one RNA selected from the group consisting of messenger RNA for expressing transcription factors which induce somatic cells and adult stem cells to be dedifferentiated into induced pluripotent stem cells, micro RNA facilitating the dedifferentiation process, and small interfering RNA.

Moreover, the present invention has the effect that the production efficiency of iPSCs can be maximized by adjusting structural properties (having the form of nanoparticles) and activity (RNA having various functions is included at the same time).

Furthermore, the present invention has the effect that low gene loading efficiency can be overcome by applying an infinite replication process to incorporate high concentrations of RNA in RNA nanoparticles.

### DESCRIPTION OF DRAWINGS

FIG. 1 is a reference diagram for explaining steps of preparing messenger RNA nanoparticles.

FIG. 2 is a reference diagram for explaining steps of preparing complex RNA nanoparticles including micro RNA and small interfering RNA.

FIG. 3 is a scanning electron microscope image of the messenger RNA nanoparticles, and a graph showing the measurement results of a nanoparticle tracking system.

FIG. 4 is optical microscope images for checking the transcription factor expression of the messenger RNA nanoparticles.

FIGS. 5 and 6 are optical microscope images for checking the iPSCs formation of the messenger RNA nanoparticles.

FIG. 7 is an image showing the result of performing a gel electrophoresis method on micro RNA nanoparticles.

FIG. 8 is a scanning electron microscope image of the micro RNA nanoparticles, and a graph showing the result of analyzing the micro RNA nanoparticles using DLS.

FIG. 9 is an image showing the result of performing the gel electrophoresis method to check the in-vivo stability of the micro RNA nanoparticles.

FIG. 10 is an image showing the result of performing the gel electrophoresis method to check the amount of micro RNA released from the micro RNA nanoparticles by a Dicer enzyme.

FIG. 11 is a graph showing the results of qPCR analysis for checking the cell transformation facilitation of complex RNA nanoparticles.

FIG. 12 is an image showing the result of performing the gel electrophoresis method to check the cell transformation facilitation of the complex RNA nanoparticles.

### MODE FOR INVENTION

Hereinafter, a method for producing induced pluripotent stem cells using RNA nanoparticles for cell transformation according to the present invention will be described in detail with reference to the drawings. Unless otherwise specified, all terms used in the present specification have the same meaning as the general meaning of the terms understood by a person with ordinary skill in the art to which the present invention belongs, and if the general meaning conflicts with the meaning of a term used in the present specification, the definition used in the present specification is applied. Moreover, detailed descriptions of well-known functions and configurations, which may unnecessarily obscure the gist of the present invention, will be omitted. In the entire specification, when it is described that one part “includes” some components, it does not mean that other components are excluded but means that other elements may be further included if there is no specific contrary description.

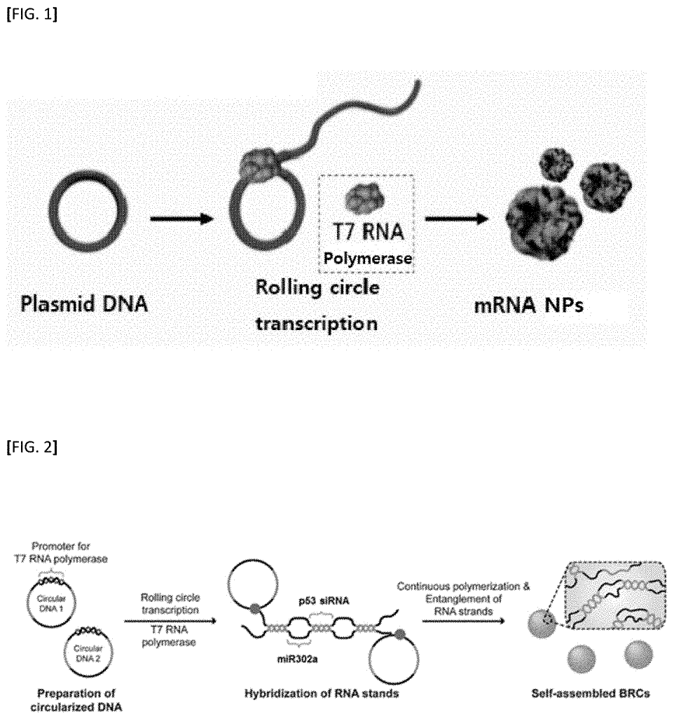

Describing the method for producing induced pluripotent stem cells using RNA nanoparticles for cell transformation according to one embodiment of the present invention with reference to FIGS. 1 to 12, the method for producing induced pluripotent stem cells includes: a particle preparation step of preparing RNA nanoparticles for cell transformation, which allow somatic cells or adult stem cells to be dedifferentiated into induced pluripotent stem cells, or facilitate the dedifferentiation; a transfer step of transferring, to the somatic cells or the adult stem cells, the RNA nanoparticles for cell transformation prepared in the particle preparation step; and a culture step of culturing the cells to which the RNA nanoparticles for cell transformation have been transferred after the transfer step to produce iPSCs.

The particle preparation step is a step for preparing RNA nanoparticles for cell transformation, which allow somatic cells or adult stem cells to be dedifferentiated into induced pluripotent stem cells, or facilitate the dedifferentiation, and RNA nanoparticles including at least one RNA selected from the group consisting of messenger RNA for expressing transcription factors which allow somatic cells or adult stem cells to be dedifferentiated into induced pluripotent stem cells, micro RNA facilitating the dedifferentiation, and small interfering RNA are prepared. The RNA nanoparticles prepared in the particle preparation step include, for example, messenger RNA nanoparticles, micro RNA nanoparticles, small interfering RNA nanoparticles, complex RNA nanoparticles including micro RNA and small interfering RNA, complex RNA nanoparticles including messenger RNA and micro RNA, complex RNA nanoparticles including messenger RNA and small interfering RNA, and complex RNA nanoparticles including messenger RNA, micro RNA, and small interfering RNA, and are introduced into somatic cells or adult stem cells to transform the cells into iPSCs or to facilitate the transformation. The RNA nanoparticles for cell transformation have a certain shape and a certain size, but preferably have a spherical shape as a whole and have a diameter of 50 to 200 nm, and consist of only biological substances and thus do not have in-vivo toxicity, and are stable in an in-vivo environment and thus can continuously release functional RNA (mRNA, miRNA, or siRNA) for a long time. The particle preparation step includes a step of preparing messenger RNA nanoparticles, a step of preparing micro RNA nanoparticles, a step of preparing small interfering RNA nanoparticles, a step of preparing complex RNA nanoparticles including micro RNA and small interfering RNA, a step of preparing complex RNA nanoparticles including messenger RNA and micro RNA, a step of preparing complex RNA nanoparticles including messenger RNA and small interfering RNA, a step of preparing complex RNA nanoparticles including messenger RNA, micro RNA, and small interfering RNA, and the like.

The step of preparing messenger RNA nanoparticles is a step of preparing messenger RNA nanoparticles including repeating messenger RNA for expressing transcription factors (for example, Oct4, Sox2, cMyc, LMyc, Klf4, Lin28, or the like) which allow somatic cells or adult stem cells to be dedifferentiated into induced pluripotent stem cells, and includes: a pDNA production step of producing circular double-stranded plasmid DNA which contains a base sequence complementary to a messenger RNA base sequence for expressing a transcription factor, a ribosome binding base sequence that is an essential element when RNA is translated into a protein, and a promoter base sequence for a T7 RNA polymerase; a particle formation step of incubating a reaction solution containing the plasmid DNA, an RNA polymerase, and the like at a certain temperature for a certain time, performing rolling circle transcription (RCT) of the plasmid DNA using the RNA polymerase to produce long single-stranded messenger RNA containing a repeating messenger RNA base sequence for expressing a transcription factor, and allowing the produced single-stranded messenger RNA to be self-assembled while being twisted and entangled, to thereby form messenger RNA nanoparticles; and the like.

The step of preparing micro RNA nanoparticles is a step of preparing micro RNA nanoparticles including repeating micro RNA (for example, miRNA-302a/b/c/d, 367, or 369, or the like) for facilitating the dedifferentiation of somatic cells or adult stem cells into induced pluripotent stem cells, and includes: a circular DNA production step of producing circular DNA containing a base sequence complementary to a micro RNA base sequence by complementarily binding a promoter to ssDNA containing the base sequence complementary to the micro RNA base sequence; a particle formation step of incubating a reaction solution containing the circular DNA, an RNA polymerase, and the like at a certain temperature for a certain time, performing rolling circle transcription (RCT) of the circular DNA using the RNA polymerase to produce long single-stranded micro RNA containing a repeating micro RNA base sequence, and allowing the produced single-stranded micro RNA to be self-assembled while being twisted and entangled, to thereby form micro RNA nanoparticles; and the like.

The step of preparing small interfering RNA nanoparticles is a step of preparing small interfering RNA nanoparticles including repeating small interfering RNA (for example, p53 siRNA or the like) for facilitating the dedifferentiation of somatic cells or adult stem cells into induced pluripotent stem cells, and includes: a circular DNA production step of producing circular DNA containing a base sequence complementary to a hairpin-shaped small interfering RNA base sequence by complementarily binding a promoter to ssDNA containing the base sequence complementary to the hairpin-shaped small interfering RNA base sequence; a particle formation step of incubating a reaction solution containing the circular DNA, an RNA polymerase, and the like at a certain temperature for a certain time, performing rolling circle transcription (RCT) of the circular DNA using the RNA polymerase to produce long single-stranded small interfering RNA containing a repeating hairpin-shaped small interfering RNA base sequence, and allowing the produced single-stranded small interfering RNA to be self-assembled while being twisted and entangled, to thereby form small interfering RNA nanoparticles; and the like. The micro RNA and/or the small interfering RNA does not cause dedifferentiation by itself, but facilitates dedifferentiation, by a conventional method or when additionally used in a method using messenger RNA nanoparticles.

The step of preparing complex RNA nanoparticles including micro RNA and small interfering RNA is a step of preparing complex RNA nanoparticles including repeating micro RNA and small interfering RNA for facilitating the dedifferentiation of somatic cells or adult stem cells into induced pluripotent stem cells, and includes: a first circular DNA production step of producing first circular DNA by complementarily binding a promoter to ssDNA which is for facilitating the dedifferentiation of somatic cells or adult stem cells into induced pluripotent stem cells, and includes a base sequence complementary to a micro RNA base sequence and a base sequence (hereinafter, referred to as a ‘si complementary base sequence’) complementary to a small interfering RNA base sequence; a second circular DNA production step of producing second circular DNA by complementarily binding a promoter to ssDNA containing a base sequence complementary to an additional micro RNA base sequence and a base sequence complementary to the si complementary base sequence; and a particle formation step of incubating a reaction solution containing the first circular DNA, the second circular DNA, an RNA polymerase, and the like at a certain temperature for a certain time, performing rolling circle transcription (RCT) of each of the first circular DNA and the second circular DNA using the RNA polymerase to form long single-stranded first RNA containing a repeating micro RNA base sequence and a small interfering RNA base sequence, and long single-stranded second RNA containing a repeating micro RNA base sequence and the base sequence complementary to the small interfering RNA base sequence, and partially complementarily binding the first RNA to the second RNA (the small interfering RNA moieties of the first RNA and the second RNA selectively form a complementary bond) to be self-assembled while being entangled, to thereby form nanoparticles.

The step of preparing complex RNA nanoparticles including messenger RNA and micro RNA is a step of preparing complex RNA nanoparticles including messenger RNA, which allow somatic cells or adult stem cells to be dedifferentiated into induced pluripotent stem cells, and micro RNA facilitating the dedifferentiation, and includes: a pDNA production step of producing circular double-stranded plasmid DNA which contains a base sequence complementary to a messenger RNA base sequence for expressing a transcription factor, a ribosome binding base sequence that is an essential element when RNA is translated into a protein, a promoter base sequence for a T7 RNA polymerase, and a base sequence (hereinafter, referred to as a ‘first binding base sequence’) that enables complementary binding to micro RNA; a circular DNA production step of producing circular DNA containing a base sequence complementary to a micro RNA base sequence and a base sequence (hereinafter, referred to as a ‘second binding base sequence’) complementary to the first binding base sequence; and a particle formation step of incubating a reaction solution containing the plasmid DNA, the circular DNA, an RNA polymerase, and the like at a certain temperature for a certain time, performing rolling circle transcription (RCT) of each of the plasmid DNA and the circular DNA using the RNA polymerase to form long single-stranded first RNA containing a repeating messenger RNA base sequence and the first binding base sequence, and long single-stranded second RNA containing a repeating micro RNA base sequence and the second binding base sequence, and partially complementarily binding the first RNA to the second RNA (the first binding base sequence of the first RNA and the second binding base sequence moiety of the second RNA selectively form a complementary bond) to be self-assembled while being entangled, to thereby form nanoparticles.

The complex RNA nanoparticles including messenger RNA and small interfering RNA can be prepared in the same manner as in the step of preparing complex RNA nanoparticles including messenger RNA and micro RNA, except that a base sequence complementary to the small interfering RNA base sequence is used instead of the base sequence complementary to the micro RNA.

In the method for preparing complex RNA nanoparticles including messenger RNA, micro RNA, and small interfering RNA, nanoparticles can be formed by preparing the plasmid DNA, the first circular DNA, and the second circular DNA, and performing rolling circle transcription using the RNA polymerase to cause self-assembly.

The transfer step is a step of transferring, to somatic cells or adult stem cells, the RNA nanoparticles for cell transformation prepared in the particle preparation step, and for example, the RNA nanoparticles for cell transformation can be transferred to cells using an electroporation method or a positively charged polymer.

The electroporation method is performed by suspending somatic cells or adult stem cells in a resuspension buffer, adding RNA nanoparticles, and then applying an electric shock using the Neon Transfection System, and the method using a positively charged polymer is performed by dispensing somatic cells or adult stem cells into a culture dish, adding a growth medium, culturing the cells for a certain time, then mixing RNA nanoparticles with a base transfection reagent (TransIT-X2 Delivery System, Mirus) that is a positively charged polymer to produce a complex, and adding the complex to a cell culture medium.

The culture step includes a culture step of culturing the cells, to which the RNA nanoparticles for cell transformation have been transferred, at a certain temperature for a certain time after the transfer step to produce iPSCs.

Another embodiment of the present invention may further include a loading step of loading, on the RNA nanoparticles, a protein that inhibits an innate immune response, between the particle preparation step and the transfer step. In the loading step, the protein can be loaded on the nanoparticles by various conventional methods, and for example, the loading can be performed by immersing the nanoparticles formed in the particle preparation step, for a certain time, in a solution in which a protein that binds to a cell membrane receptor and inhibits an innate immune response based on an IFN-β signaling pathway is dispersed.

Hereinafter, the present invention will be described in more detail with reference to Examples. However, these Examples are merely for describing the present invention in more detail, and the scope of the present invention is not limited to these Examples.

### <Example 1> Preparation of Messenger RNA Nanoparticles for Cell Transformation

1. Plasmid DNA
(SEQ ID NO: 1)
(TCGCGCGTTTCGGTGATGACGGTGAAAACCTCTGACACATGCAGCTCCCGGAGACGGTCAC
AGCTTGTCTGTAAGCGGATGCCGGGAGCAGACAAGCCCGTCAGGGCGCGTCAGCGGGTGTTG
GCGGGTGTCGGGGCTGGCTTAACTATGCGGCATCAGAGCAGATTGTACTGAGAGTGCACCAA
ATGCGGTGTGAAATACCGCACAGATGCGTAAGGAGAAAATACCGCATCAGGCGCCATTCGCC
ATTCAGGCTGCGCAACTGTTGGGAAGGGCGATCGGTGCGGGCCTCATCGCTATTACGCCAGC
TGGCGAAAGGGGGATGTGCTGCAAGGCGATTAAGTTGGGTAACGCCAGGGTTTTCCCAGTCA
CGACGTTGTAAAACGACGGCCAGTGCAACGCGATGACGATGGATAGCGATTCATCGATGAGC
TGACCCGATCGCCGCCGCCGGAGGGTTGCGTTTGAGACGGGCGACAGATGAGGCTCGTTTAG
TGAACCGTCAGATCGCCTGGAGACGCCATCCACGCTGTTTTGACCTCCATAGAAGACACCGG
GACCGATCCAGCCTCCGGACTCTAGAGGATCGAACCCTTTTGGACCCTCGTACAGAAGC-
TAATACGACTCACTATAG -
(SEQ ID NO: 2)
GGAAATAAGAGAGAAAAGAAGAGTAAGAAGAAATATAAGA-GCCACCATGG -
ATGGCGGcccGACACCTGGCTTCGGATTTCGCCTTCTCGCCCCCTCCAGGTGGTGGAGGTGA
TGGGCCAGGGGGGCCGGAGCCGGGCTGGGTTGATCCTCGGACCTGGCTAAGCTTCCAAGGCC
CTCCTGGAGGGCCAGGAATCGGGCCGGGGGTTGGGCCAGGCTCTGAGGTGTGGGGGATTCCC
CCATGCCCCCCGCCGTATGAGTTCTGTGGGGGGATGGCGTACTGTGGGCCCCAGGTTGGAGT
GGGGCTAGTGCCCCAAGGCGGCTTGGAGACCTCTCAGCCTGAGGGCGAAGCAGGAGTCGGGG
TGGAGAGCAACTCCGATGGGGCCTCCCCGGAGCCCTGCACCGTCACCCCTGGTGCCGTGAAG
CTGGAGAAGGAGAAGCTGGAGCAAAACCCGGAGGAGTCCCAGGACATCAAAGCTCTGCAGAA
AGAACTCGAGCAATTTGCCAAGCTCCTGAAGCAGAAGAGGATCACCCTGGGATATACACAGG
CCGATGTGGGGCTCACCCTGGGGGTTCTATTTGGGAAGGTATTCAGCCAAACGACCATCTGC
CGCTTTGAGGCTCTGCAGCTTAGCTTCAAGAACATGTGTAAGCTGCGGCCCTTGCTGCAGAA
GTGGGTGGAGGAAGCTGACAACAATGAAAATCTTCAGGAGATATGCAAAGCAGAAACCCTCG
TGCAGGCCCGAAAGAGAAAGCGAACCAGTATCGAGAACCGAGTGAGAGGCAACCTGGAGAAT
TTGTTCCTGCAGTGCCCGAAACCCACACTGCAGCAGATCAGCCACATCGCCCAGCAGCTTGG
GCTCGAGAAGGATGTGGTCCGAGTGTGGTTCTGTAACCGGCGCCAGAAGGGCAAGCGATCAA
GCAGCGACTATGCACAACGAGAGGATTTTGAGGCTGCTGGGTCTCCTTTCTCAGGGGGACCA
GTGTCCTTTCCTCTGGCCCCAGGGCCCCATTTTGGTACCCCAGGCTATGGGAGCCCTCACTT
CACTGCACTGTACTCCTCGGTCCCTTTCCCTGAGGGGGAAGCCTTTCCCCCTGTCTCTGTCA
(SEQ ID NO: 3 )
CCACTCTGGGCTCTCCCATGCATTCAAACTGA -
ATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCTAGAT
CCTTTTAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTTCATCC
ATAGTTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAATAAACC
AGCCAGCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCA
ATACGGGATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACA
GGAAGGCAAAATGCCGCAAAAAAGGGAATAAGGGCGACACGGAAATGTTGAATACTCATACT
CTACCTTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAAAGTGCCA
CCTGACGTCTAAGAAACCATTATTATCATGACATTAACCTATAAAAATAGGCGTATCACGAG
GCCCTTTCGTC)

containing: a promoter base sequence (SEQ ID NO: 1) for a T7 RNA polymerase; a ribosome binding base sequence (SEQ ID NO: 2) that is an essential element when RNA is translated into a protein; and a base sequence (SEQ ID NO: 3) complementary to a messenger RNA base sequence for expressing Oct4 playing a key role among transcription factors, was designed.

2. 1 nM of the plasmid DNA, 1 mM of a ribonucleotide solution mix (New England Biolabs), a reaction buffer (8 mM of Tris-HCl, 0.4 mM of spermidine, 1.2 mM of MgCl₂, and 2 mM of dithiothreitol), and 50 units ml⁻¹ of a T7 RNA polymerase (New England Biolabs) were added to a tube and mixed, the tube was placed in an incubator, and the mixture was reacted at 37° C. for 20 hours to produce messenger RNA nanoparticles for cell transformation.

### <Example 2> Checking of Shape, Size, and Distribution of Messenger RNA Nanoparticles for Cell Transformation

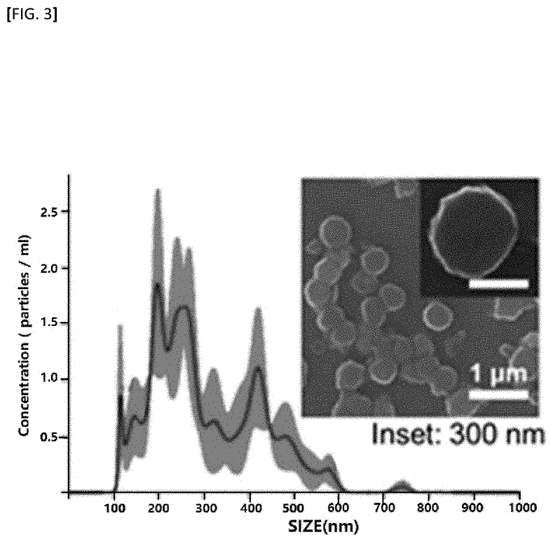

The results of measuring the size and shape of the messenger RNA nanoparticles for cell transformation produced in Example 1 using a scanning electron microscope are shown in FIG. 3, and the results of measurement using a nanoparticle tracking system (NTA; nanoparticle tracking analysis) by which the concentration, size, and size distribution of nanoparticles can be checked by recording Brownian motion of individual particles are also shown in FIG. 3. From FIG. 3, nanoparticles having a diameter of 100 to 200 nm and having a spherical shape can be confirmed.

### <Example 3> Checking of Transcription Factor Expression of Messenger RNA Nanoparticles for Cell Transformation

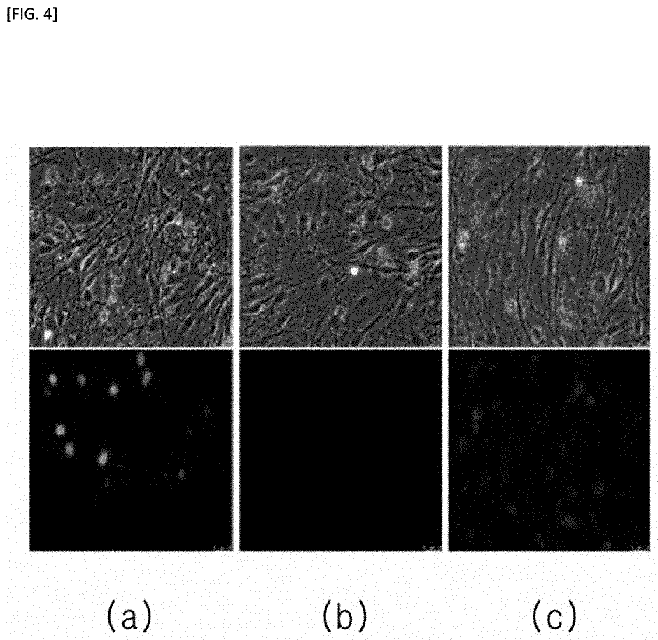

1. Human fibroblasts (human dermal fibroblasts, HDFs) were suspended in a resuspension buffer R (Thermo Fisher), plasmid vectors encoding specific transcription factors were added, then an electric shock (the pulse voltage, the pulse width, and the pulse number were 1650 V, 10 ms, and 3, respectively) was applied using the Neon Transfection System (Thermo Fisher), the HDFs subjected to the electroporation were dispensed into a gelatin-coated culture dish, the resultant was treated with a complex formed by mixing the messenger RNA nanoparticles for cell transformation produced in Example 1 with a TransIT-X2 reagent, and then Oct4 protein expression was checked using an immunostaining method. The Oct4 protein expression was measured using an optical microscope 48 hours after the final transfer of the vectors or the nanoparticles, and the results thereof are shown in FIG. 4. (a) of FIG. 4 shows the results of measuring HDFs to which plasmid vectors encoding transcription factors Oct4, Sox2, Klf4, L-Myc, and Lin28 were transferred, (b) of FIG. 4 shows the results of measuring HDFs to which plasmid vectors encoding transcription factors Sox2, Klf4, L-Myc, and Lin28 were transferred, and (c) of FIG. 4 shows the results of measuring HDFs to which plasmid vectors encoding Sox2, Klf4, L-Myc, and Lin28 were transferred and then the messenger RNA nanoparticles for cell transformation produced in Example 1 were additionally transferred.

2. As shown in FIG. 4, unlike the case where the plasmid vectors encoding Sox2, Klf4, L-Myc, and Lin28 were transferred (see (b) of FIG. 4), in the case where the messenger RNA nanoparticles were additionally transferred (see (c) of FIG. 4), the expression (green fluorescence) of Oct4 was observed similarly to the case where Oct4 was transferred in the form of a plasmid vector (see (a) of FIG. 4), and thus it can be seen that the messenger RNA nanoparticles for cell transformation express transcription factors.

### <Example 4> Checking 1 of iPSCs Formation Through Transfer of Messenger RNA Nanoparticles for Cell Transformation

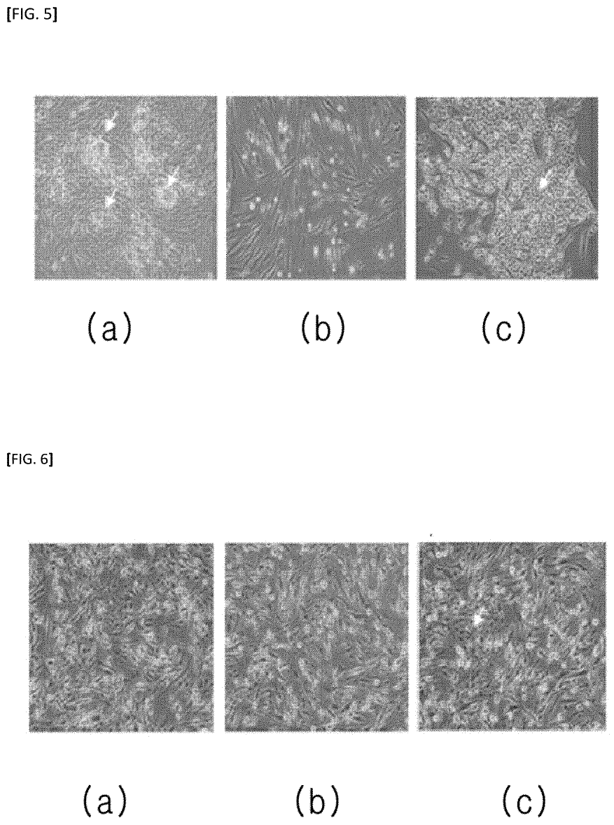

1. The HDFs to which the messenger RNA nanoparticles (18 ug) produced in Example 1, together with the plasmid vectors encoding transcription factors, were transferred using an electroporation method were cultured in a DMEM (growth medium) containing 10% fetal bovine serum (FBS), the HDFs were dispensed into a Matrigel-coated culture dish, an E8 medium was replaced daily, and iPSCs formation was checked. The iPSCs formation was measured using an optical microscope 10 days after the final transfer of the vectors or the nanoparticles, and the results thereof are shown in FIG. 5. (a) of FIG. 5 shows the results of measuring HDFs to which plasmid vectors encoding transcription factors Oct4, Sox2, Klf4, L-Myc, and Lin28 were transferred, (b) of FIG. 5 shows the results of measuring HDFs to which plasmid vectors encoding transcription factors Sox2, Klf4, L-Myc, and Lin28 were transferred, and (c) of FIG. 5 shows the results of measuring HDFs to which the messenger RNA nanoparticles for cell transformation were additionally transferred together with plasmid vectors encoding Sox2, Klf4, L-Myc, and Lin28.

2. As shown in FIG. 5, unlike the case where the plasmid vectors encoding Sox2, Klf4, L-Myc, and Lin28 were transferred (see (b) of FIG. 5), in the case where the messenger RNA nanoparticles were additionally transferred (see (c) of FIG. 5), it can be confirmed that fake colonies, which are modified cell populations, were observed during the dedifferentiation process (see the arrows in (a) and (c) of FIG. 5) similarly to the case where Oct4 was transferred in the form of a plasmid vector (see (a) of FIG. 5), and thus it can be seen that induced pluripotent stem cells can be produced using the messenger RNA nanoparticles for cell transformation.

### <Example 5> Checking 2 of iPSCs Formation Through Transfer of Messenger RNA Nanoparticles for Cell Transformation

1. The HDFs to which the messenger RNA nanoparticles (30 ug) produced in Example 1, together with the plasmid vectors encoding transcription factors, were transferred using an electroporation method were cultured in a DMEM (growth medium) containing 10% fetal bovine serum (FBS), the HDFs were dispensed onto MEF feeder cells, an E8 medium was replaced daily, and iPSCs formation was checked. The iPSCs formation was measured using an optical microscope 5 days after the final transfer of the vectors or the nanoparticles, and the results thereof are shown in FIG. 6. (a) of FIG. 6 shows the results of measuring HDFs to which plasmid vectors encoding transcription factors Oct4, Sox2, Klf4, L-Myc, and Lin28 were transferred, (b) of FIG. 6 shows the results of measuring HDFs to which plasmid vectors encoding transcription factors Sox2, Klf4, L-Myc, and Lin28 were transferred, and (c) of FIG. 6 shows the results of measuring HDFs to which the messenger RNA nanoparticles for cell transformation were additionally transferred together with plasmid vectors encoding Sox2, Klf4, L-Myc, and Lin28.

2. As shown in FIG. 6, unlike the case where the plasmid vectors encoding Sox2, Klf4, L-Myc, and Lin28 were transferred (see (b) of FIG. 6), in the case where the messenger RNA nanoparticles were additionally transferred (see (c) of FIG. 6), it can be confirmed that mesenchymal-epithelial transition (MET) observed during the dedifferentiation process was observed (see the arrow in (c) of FIG. 6) similarly to the case where Oct4 was transferred in the form of a plasmid vector (see (a) of FIG. 6), and thus it can be seen that induced pluripotent stem cells can be produced using the messenger RNA nanoparticles for cell transformation.

3. Meanwhile, Oct4 is a transcription factor (master regulator) that plays a key role in dedifferentiation, and has been known in several studies to be irreplaceable and to be maintained in a relatively high amount during the dedifferentiation process, compared to other factors such as Sox2, cMyc, Klf4, and Lin28, and through Examples 1 to 5, it was confirmed that dedifferentiation is possible by transferring Oct4 in the form of messenger RNA nanoparticles. Therefore, it is considered that iPSCs can be produced by transferring all of the remaining transcription factors in the form of messenger RNA nanoparticles.

### <Example 6> Production and Characteristic Checking of Micro RNA Nanoparticles for Cell Transformation

1. Production of Micro RNA 34a Nanoparticles

(1) ssDNA [5′-Phosphate-ATAGTGAGTCGTATTA (SEQ ID NO: 4)-ACGTACCAAAGGGCAGTATACTTGCTGATTGTTACTTGAAACAACCAGCTAAGACACTGCCA TTGAGGCA (SEQ ID NO: 5)-ATCCCT (SEQ ID NO: 6)-3′] containing: base sequences that enable complementary binding to a promoter, at both ends; and a base sequence (SEQ ID NO: 5) complementary to a micro RNA 34a base sequence, in the center, was designed. Moreover, a promoter [5′-TAATACGACTCACTATAGGGAT-3′ (SEQ ID NO: 7)] was designed.

(2) 1 μM of the ssDNA and 1 μM of the promoter DNA were mixed with nuclease free water, and the mixture was heated at 95° C. for 2 minutes using a PCR thermal cycler, and gradually cooled to 25° C. for 1 hour. In the process, both ends of the ssDNA were complementarily bound to the promoter DNA to form circular DNA. Thereafter, in order to ligate nicks in the circular DNA, 0.06 Uμl⁻¹ of a T4 ligase and a ligase buffer (50 mM of Tris-HCl, 10 mM of MgCl₂, 10 mM of DTT, and 1 mM of ATP) were mixed and cultured overnight at room temperature to produce a complete circular DNA.

(3) 0.03 μM of the circular DNA, 2 mM of each ribonucleotide solution mix (25 mM of each NTP), a 2× reaction buffer (400 mM of Tris-HCl, 20 mM of spermidine, 60 mM of MgCl₂, and 100 mM of dithiothreitol), and 5 units μl⁻¹ of a T7 RNA polymerase were mixed to produce a reaction solution, and the reaction solution was cultured at a temperature of 37° C. for 20 hours to produce micro RNA nanoparticles (miR34a-NP).

2. Production of Micro RNA 302a Nanoparticles

Micro RNA nanoparticles were produced under the same conditions as in section 1 of Example 6, except that ssDNA [5′-Phosphate-ATAGTGAGTCGTATTA (SEQ ID NO: 4)-ACTCCTACTAAAACATGGAAGCACTTACTTTTAAAGTCACAGAAAGCACTTCCATGTTAAAG TTGAAGGGAGC (SEQ ID NO: 8)-ATCCCT (SEQ ID NO: 6)-3′] containing: base sequences that enable complementary binding to a promoter at both ends; and a base sequence (SEQ ID NO: 8) complementary to a micro RNA 302a base sequence, in the center, was used.

3. Characteristic Checking of Micro RNA

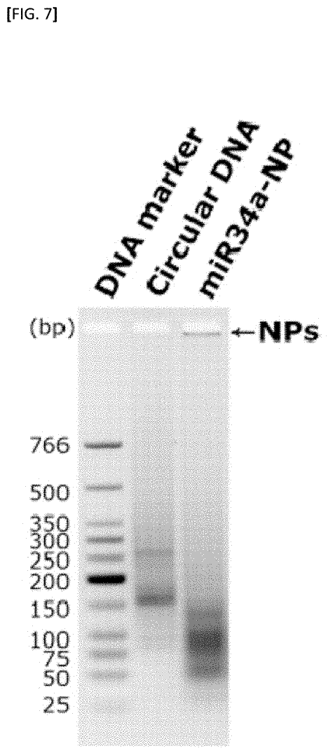

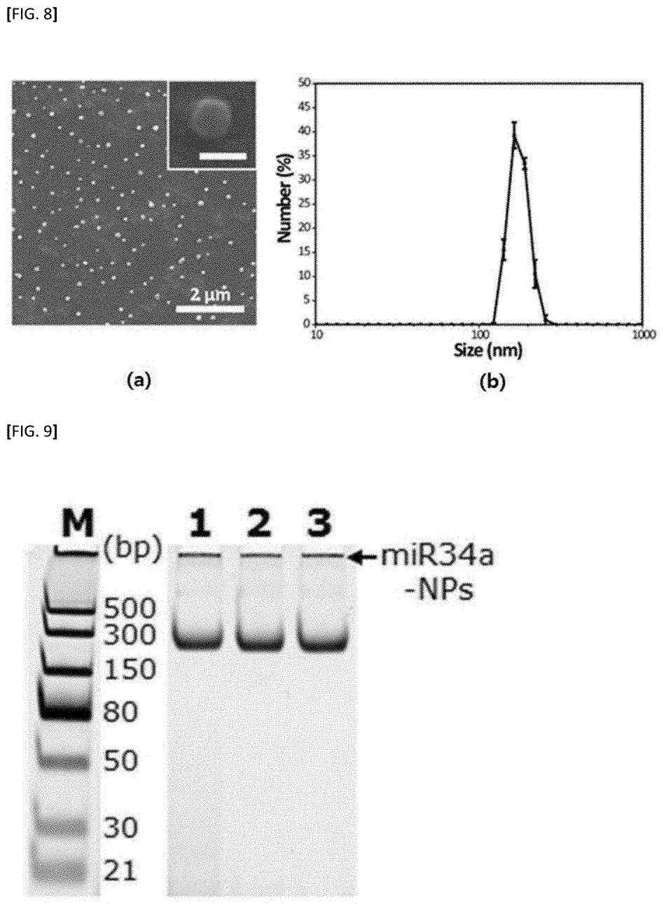

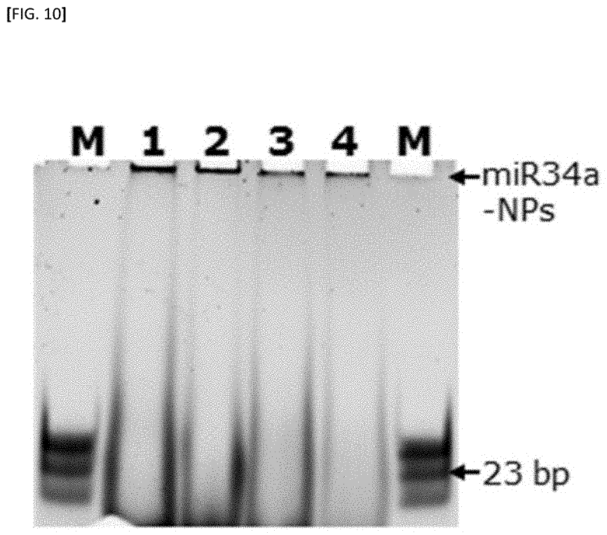

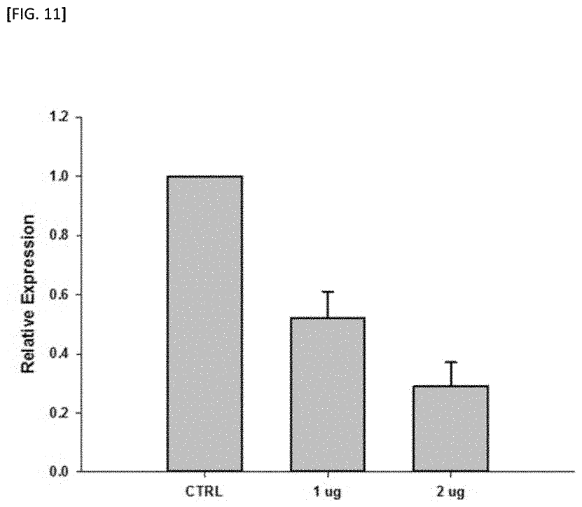

(1) A gel electrophoresis method was performed on the circular DNA and the micro RNA nanoparticles (miR34a-NP), which were produced in section 1 of Example 6, and the results thereof are shown in FIG. 7. Moreover, for the micro RNA nanoparticles produced in section 1 of Example 6, the result of checking the micro RNA nanoparticles using a scanning electron microscope is shown in (a) of FIG. 8, and the result of analyzing the micro RNA nanoparticles using dynamic light scattering (DLS) is shown in (b) of FIG. 8. Further, in order to check the in-vivo stability of the micro RNA, the micro RNA nanoparticles produced in section 1 of Example 6 were incubated in a cell culture environment containing 10% FBS, and then a gel electrophoresis method was performed, and the results thereof are shown in FIG. 9 (in FIG. 9, M represents the result of dsRNA ladder, 1 represents the result of culturing miR34a-NP for 0 hours (band intensity is 1.00), 2 represents the result of culturing miR34a-NP for 12 hours (band intensity is 0.81), and 3 represents the result of culturing miR34a-NP for 24 hours (band intensity is 0.87)). Furthermore, in order to check that the micro RNA nanoparticles were cut by a Dicer enzyme to release functional micro RNA, the micro RNA nanoparticles produced in section 1 of Example 6 and Dicer were incubated together, and then a gel electrophoresis method was performed, and the results thereof are shown in FIG. 10. (in FIG. 10, M represents the result of a siRNA marker, 1 represents the result of culturing miR34a-NP for 0 hours (band intensity is 1.00), 2 represents the result of culturing miR34a-NP for 12 hours (band intensity is 0.80), 3 represents the result of culturing miR34a-NP for 24 hours (band intensity is 0.67), and 4 represents the result of culturing miR34a-NP for 48 hours (band intensity is 0.55)).

(2) It can be confirmed from FIG. 7 that multimeric micro RNA was self-assembled in the form of nanoparticles to significantly increase the molecular weight, it can be confirmed from FIG. 8 that the micro RNA nanoparticles had a size of about 100 nm for easy endocytosis and had a spherical shape with a smooth surface, and a dispersity value was about 0.2, which indicates that the particles had an even size distribution, and it can be confirmed from FIG. 9 that RNA strands were broken down in minutes in an in-vivo environment, but 80% or more of the micro RNA particles were not broken down after 24 hours, and a high molecular weight state was maintained. Moreover, it can be confirmed from FIG. 10 that the micro RNA nanoparticles released RNA over time after the treatment with the Dicer enzyme, and the molecular weight thereof was decreased, and in particular, it can be seen that about 40% or more of the micro RNA nanoparticles maintained a high molecular weight even after 48 hours had elapsed, which indicates that the micro RNA nanoparticles can continuously release micro RNA for a long time in an in-vivo environment.

### <Example 7> Production of Complex RNA Nanoparticles for Cell Transformation

1. Production of Complex RNA Nanoparticles Including miRNA-302a and p53 siRNA

(1) Circular DNA (first circular DNA) was produced under the same conditions as in parts (1) and (2) of section 1 of Example 6, except that ssDNA [5′-Phosphate-ATAGTGAGTCGTATTA (SEQ ID NO: 4)-AAGTAGATTACCACTGGAGTCTT (SEQ ID NO: 9)-AGCAAGTACATCCACGTTTAAGT (SEQ ID NO: 10)-AAGTAGATTACCACTGGAGTCTT (SEQ ID NO: 9)-ATCCCT (SEQ ID NO: 6)-3′] containing: base sequences that enable complementary binding to a promoter, at both ends; and a base sequence (SEQ ID NO: 10) complementary to a micro RNA 302a-5p base sequence between two repeated base sequences (si complementary base sequence, SEQ ID NO: 9) complementary to a siRNA p53 base sequence, in the center, was used.

(2) Circular DNA (second circular DNA) was produced under the same conditions as in parts (1) and (2) of section 1 of Example 6, except that ssDNA [5′-Phosphate-ATAGTGAGTCGTATTA (SEQ ID NO: 4)-AAGACTCCAGTGGTAATCTACTT (SEQ ID NO: 11)-TCACCAAAACATGGAAGCACTTA (SEQ ID NO: 12)-AAGACTCCAGTGGTAATCTACTT (SEQ ID NO: 11)-ATCCCT (SEQ ID NO: 6)-3′] containing: base sequences that enable complementary binding to a promoter, at both ends; and a base sequence (SEQ ID NO: 12) complementary to a micro RNA 302a-3p base sequence between two repeated base sequences (SEQ ID NO: 11) complementary to a si complementary base sequence, in the center, was used.

(3) 2.5 μM of the first circular DNA, 2.5 μM of the second circular DNA, 2 mM of each ribonucleotide solution mix (25 mM of each NTP), a 2× reaction buffer (400 mM of Tris-HCl, mM of spermidine, 60 mM of MgCl₂, and 100 mM of dithiothreitol), and 80 units μl⁻¹ of a T7 RNA polymerase were mixed to produce a reaction solution, and the reaction solution was cultured at a temperature of 37° C. for 20 hours to produce complex RNA nanoparticles including micro RNA and siRNA.

2. Production of Complex RNA Nanoparticles Including miRNA-302a/b/c/d, 367, and 369 and p53 siRNA

(1) Complex RNA nanoparticles including micro RNA and small interfering RNA were produced under the same conditions as in part (1) of Example 7, except that circular DNA (first circular DNA) was produced using ssDNA [5′-Phosphate-ATAGTGAGTCGTATTA (SEQ ID NO: 4)-AAGTAGATTACCACTGGAGTCTT (SEQ ID NO: 9)-TCACCAAAACATGGAAGCACTTA (SEQ ID NO: 12)-AAGTAGATTACCACTGGAGTCTT (SEQ ID NO: 9)-CTACTAAAACATGGAAGCACTTA (SEQ ID NO: 13)-AAGTAGATTACCACTGGAGTCTT (SEQ ID NO: 9)-TCACCATTGCTAAAGTGCAATTC (SEQ ID NO: 14)-AAGTAGATTACCACTGGAGTCTT (SEQ ID NO: 9)-ATCCCT (SEQ ID NO: 6)-3′] in which base sequences that enable complementary binding to a promoter, at both ends; a base sequence (siP53-sense complementary base sequence) complementary to a siRNA p53-sense base sequence; a base sequence (SEQ ID NO: 12) complementary to a micro RNA 302a-3p base sequence; a siP53-sense complementary base sequence; a base sequence (SEQ ID NO: 13) complementary to a micro RNA 302b base sequence; a siP53-sense complementary base sequence; a base sequence (SEQ ID NO: 14) complementary to a micro RNA 367 base sequence; and a siP53-sense complementary base sequence are repeated in sequence, and circular DNA (second circular DNA) was produced using ssDNA [5′-Phosphate-ATAGTGAGTCGTATTA (SEQ ID NO: 4)-AAGACTCCAGTGGTAATCTACTT (SEQ ID NO: 11)-ACACTCAAACATGGAAGCACTTA (SEQ ID NO: 15)-No:AAGACTCCAGTGGTAATCTACTT (SEQ ID NO: 11)-GAAAAGATCAACCATGTATTATT (SEQ ID NO: 16)-AAGACTCCAGTGGTAATCTACTT (SEQ ID NO: 11)-CCACTGAAACATGGAAGCACTTA (SEQ ID NO: 17)-AAGACTCCAGTGGTAATCTACTT (SEQ ID NO: 11)-ATCCCT (SEQ ID NO: 6)-3′] in which base sequences that enable complementary binding to a promoter, at both ends; a base sequence (siP53-antisense complementary base sequence) complementary to a siRNA p53-antisense base sequence; a base sequence (SEQ ID NO: 15) complementary to a micro RNA 302d base sequence; a siP53-antisense complementary base sequence; a base sequence (SEQ ID NO: 16) complementary to a micro RNA 369 base sequence; a siP53-antisense complementary base sequence; a base sequence (SEQ ID NO: 17) complementary to a micro RNA 302c base sequence; and a siP53-antisense complementary base sequence are repeated in sequence. Here, in order to maintain functionality, the micro RNA moieties except for the small interfering RNA were arranged so as to minimize complementary binding.

### <Example 8> Checking 1 of Cell Transformation Facilitation of Complex RNA Nanoparticles

1. After HDFs were dispensed into a culture dish, when the confluency reached about 70%, a treatment with a complex formed by mixing the complex RNA nanoparticles produced in section 1 of Example 7 with a TransIT-X2 reagent that is a positively charged polymer was performed for 24 hours, the resultant was transferred into cells, the cells were collected 72 hours after replacement with the growth medium, total RNA was isolated, and then the expression level of a p53 gene was analyzed through a qPCR method, and the results thereof are shown in FIG. 11. The expression level of the p53 gene was normalized with the expression level of β-actin that is a house-keeping gene, and comparison was performed.

2. It can be confirmed from FIG. 11 that after the complex RNA nanoparticles were transferred to the HDFs, siRNA was released together with a micro RNA monomer by an intracellular mechanism to cause a gene expression inhibition phenomenon. Since it is known that inhibiting the expression of p53 improves the dedifferentiation efficiency of the cells, it can be seen that the complex RNA nanoparticles can facilitate cell transformation.

### <Example 9> Checking 2 of Cell Transformation Facilitation of Complex RNA Nanoparticles

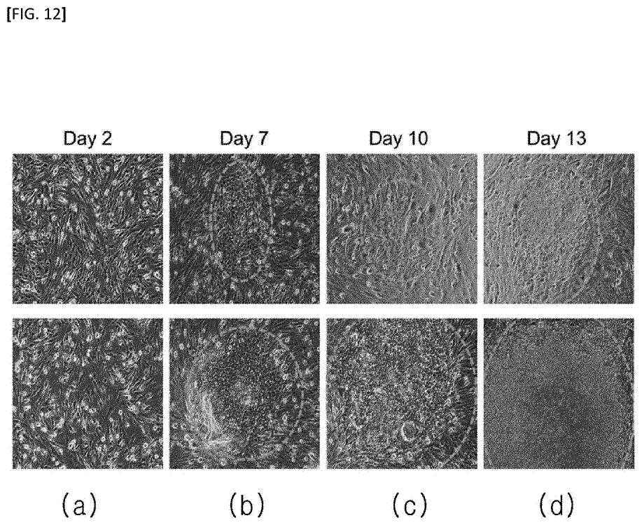

1. Conventional plasmid DNA capable of expressing a Yamanaka factor was inserted into HDFs through an electroporation method, after 24 hours, a treatment with the complex RNA nanoparticles (1 ug/ml) coated with TransIT-X2 and produced in section 2 of Example 7 was performed for 4 hours, and after a recovery period of 4 days, the cells were placed on a MEF feeder. One day later, the culture medium of the cells was exchanged with an E8 medium, and then the transformation process of the cells over time was checked using an optical microscope, and the results thereof are shown in FIG. 12. (a) of FIG. 12 shows the result of using only the conventional plasmid DNA, and (b) of FIG. 12 shows the result of additionally treating the conventional plasmid DNA with complex RNA nanoparticles.

2. It can be confirmed from FIG. 12 that iPSC colonies were more efficiently produced within a short time due to the action of the released p53 siRNA and six kinds of micro RNA, compared to the control group. Consequently, it can be seen that the complex RNA nanoparticles can facilitate cell transformation.

Hereinbefore, the applicant has described the preferred embodiments of the present invention, but these embodiments are merely one embodiment which implements the technical idea of the present invention, and any change examples or modification examples should be interpreted as falling within the scope of the present invention as long as the technical idea of the present invention is implemented.

1. A method for producing induced pluripotent stem cells, the method comprising:
a transfer step of transferring, to somatic cells or adult stem cells, RNA nanoparticles for cell transformation, which allow the somatic cells or the adult stem cells to be dedifferentiated into induced pluripotent stem cells, or facilitate the dedifferentiation; and
a culture step of culturing the cells to which the RNA nanoparticles for cell transformation have been transferred after the transfer step to produce induced pluripotent stem cells.

2. The method for producing induced pluripotent stem cells of claim 1, wherein the RNA nanoparticles for cell transformation include at least one RNA selected from the group consisting of messenger RNA for expressing transcription factors which allow somatic cells or adult stem cells to be dedifferentiated into induced pluripotent stem cells, micro RNA facilitating the dedifferentiation, and small interfering RNA.

3. The method for producing induced pluripotent stem cells of claim 1, wherein at least one RNA nanoparticle selected from the group consisting of messenger RNA nanoparticles for expressing transcription factors which allow somatic cells or adult stem cells to be dedifferentiated into induced pluripotent stem cells, micro RNA nanoparticles facilitating the dedifferentiation, small interfering RNA nanoparticles facilitating the dedifferentiation, complex RNA nanoparticles which include micro RNA facilitating the dedifferentiation and small interfering RNA facilitating the dedifferentiation, complex RNA nanoparticles which include messenger RNA for expressing a transcription factor and micro RNA facilitating the dedifferentiation, complex RNA nanoparticles which include messenger RNA for expressing a transcription factor and small interfering RNA facilitating the dedifferentiation, and complex RNA nanoparticles which include messenger RNA for expressing a transcription factor, micro RNA facilitating the dedifferentiation, and small interfering RNA facilitating the dedifferentiation is used as the RNA nanoparticles for cell transformation.

4. The method for producing induced pluripotent stem cells of claim 1, wherein the RNA nanoparticles for cell transformation each have a spherical shape and have a diameter of 50 to 200 nm.

5. The method for producing induced pluripotent stem cells of claim 1, wherein the RNA nanoparticles for cell transformation include messenger RNA nanoparticles for expressing transcription factors which allow somatic cells or adult stem cells to be dedifferentiated into induced pluripotent stem cells, complex RNA nanoparticles which include micro RNA facilitating the dedifferentiation and small interfering RNA facilitating the dedifferentiation, and complex RNA nanoparticles which include messenger RNA for expressing a transcription factor and micro RNA facilitating the dedifferentiation.

6. The method for producing induced pluripotent stem cells of claim 5, wherein the messenger RNA nanoparticles are produced through a pDNA production step of producing plasmid DNA containing a base sequence complementary to a repeating messenger RNA base sequence for expressing transcription factors which allow somatic cells or adult stem cells to be dedifferentiated into induced pluripotent stem cells; and a particle formation step of incubating a reaction solution containing the plasmid DNA and an RNA polymerase at a certain temperature for a certain time, performing rolling circle transcription of the plasmid DNA using the RNA polymerase to produce long single-stranded messenger RNA containing the repeating messenger RNA base sequence for expressing transcription factors, and allowing the produced single-stranded messenger RNA to be self-assembled while being twisted and entangled, to thereby form nanoparticles.

7. The method for producing induced pluripotent stem cells of claim 5, wherein the complex RNA nanoparticles, which include micro RNA facilitating the dedifferentiation and small interfering RNA facilitating the dedifferentiation, are produced through a first circular DNA production step of producing first circular DNA by complementarily binding a promoter to ssDNA which is for facilitating the dedifferentiation of somatic cells or adult stem cells into induced pluripotent stem cells, and includes a base sequence complementary to a micro RNA base sequence and a si complementary base sequence that is a base sequence complementary to a small interfering RNA base sequence; a second circular DNA production step of producing second circular DNA by complementarily binding a promoter to ssDNA containing a base sequence complementary to a micro RNA base sequence and a base sequence complementary to the si complementary base sequence; and a particle formation step of incubating a reaction solution containing the first circular DNA, the second circular DNA, and an RNA polymerase at a certain temperature for a certain time, performing rolling circle transcription of each of the first circular DNA and the second circular DNA using the RNA polymerase to form long single-stranded first RNA containing a repeating micro RNA base sequence and a small interfering RNA base sequence, and long single-stranded second RNA containing a repeating micro RNA base sequence and the base sequence complementary to the small interfering RNA base sequence, and partially complementarily binding the first RNA to the second RNA to be self-assembled while being entangled, to thereby form nanoparticles.

8. The method for producing induced pluripotent stem cells of claim 5, wherein the complex RNA nanoparticles, which include messenger RNA for expressing a transcription factor and micro RNA facilitating the dedifferentiation, are produced through a pDNA production step of producing plasmid DNA containing a base sequence complementary to a messenger RNA base sequence for expressing a transcription factor and a first binding base sequence that is a base sequence that enables complementary binding to micro RNA; a circular DNA production step of producing circular DNA containing a base sequence complementary to a micro RNA base sequence and a base sequence complementary to the first binding base sequence; and a particle formation step of incubating a reaction solution containing the plasmid DNA, the circular DNA, and an RNA polymerase at a certain temperature for a certain time, performing rolling circle transcription of each of the plasmid DNA and the circular DNA using the RNA polymerase to form long single-stranded first RNA containing a repeating messenger RNA base sequence and the first binding base sequence, and long single-stranded second RNA containing a repeating micro RNA base sequence and a second binding base sequence, and partially complementarily binding the first RNA to the second RNA to be self-assembled while being entangled, to thereby form nanoparticles.

9. The method for producing induced pluripotent stem cells of claim 5, further comprising a loading step of loading, on the RNA nanoparticles, a protein that inhibits an innate immune response, before the transfer step.

10. The method for producing induced pluripotent stem cells of claim 5, wherein in the transfer step, an electroporation method or a method using a positively charged polymer is used,
the electroporation method is performed by suspending somatic cells or adult stem cells in a resuspension buffer, adding RNA nanoparticles, and then applying an electric shock, and
the method using a positively charged polymer is performed by dispensing somatic cells or adult stem cells into a culture dish, adding a growth medium, culturing the cells for a certain time, and then adding, to the growth medium, a complex formed by mixing RNA nanoparticles with a positively charged polymer.

11. The method for producing induced pluripotent stem cells of claim 2, wherein the RNA nanoparticles for cell transformation each have a spherical shape and have a diameter of 50 to 200 nm.

12. The method for producing induced pluripotent stem cells of claim 3, wherein the RNA nanoparticles for cell transformation each have a spherical shape and have a diameter of 50 to 200 nm.


SEQUENCE LISTING (17 sequences)

SEQ ID NO: 1 - DNA, 18 bp, Artificial Sequence
  promoter region of plasmid DNA for RNA polymerase
taatacgactcactatag

SEQ ID NO: 2 - DNA, 10 bp, Artificial Sequence
  region of plasmid DNA for binding ribosome
gccaccatgg

SEQ ID NO: 3 - DNA, 1083 bp, Artificial Sequence
  region of plasmid DNA for Oct4 generation
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactga

SEQ ID NO: 4 - DNA, 16 bp, Artificial Sequence
  region of ssDNA for binding promoter DNA
atagtgagtcgtatta

SEQ ID NO: 5 - DNA, 70 bp, Artificial Sequence
  region of ssDNA for miRNA 34a generation
acgtaccaaagggcagtatacttgctgattgttacttgaaacaaccagctaagacactgccattgaggca

SEQ ID NO: 6 - DNA, 6 bp, Artificial Sequence
  region of ssDNA for binding promoter DNA
atccct

SEQ ID NO: 7 - DNA, 22 bp, Artificial Sequence
  promoter DNA for circular DNA generation
taatacgactcactatagggat

SEQ ID NO: 8 - DNA, 73 bp, Artificial Sequence
  region of ssDNA for miRNA 302a generation
actcctactaaaacatggaagcacttacttttaaagtcacagaaagcacttccatgttaaagttgaagggagc

SEQ ID NO: 9 - DNA, 23 bp, Artificial Sequence
  region of ssDNA for siRNA p53 generation
aagtagattaccactggagtctt

SEQ ID NO: 10 - DNA, 23 bp, Artificial Sequence
  region of ssDNA for miRNA 302a-5p generation
agcaagtacatccacgtttaagt

SEQ ID NO: 11 - DNA, 23 bp, Artificial Sequence
  region of ssDNA for siRNA p53 generation
aagactccagtggtaatctactt

SEQ ID NO: 12 - DNA, 23 bp, Artificial Sequence
  region of ssDNA for miRNA 302a-3p generation
tcaccaaaacatggaagcactta

SEQ ID NO: 13 - DNA, 23 bp, Artificial Sequence
  region of ssDNA for miRNA 302b generation
ctactaaaacatggaagcactta

SEQ ID NO: 14 - DNA, 23 bp, Artificial Sequence
  region of ssDNA for miRNA 367 generation
tcaccattgctaaagtgcaattc

SEQ ID NO: 15 - DNA, 23 bp, Artificial Sequence
  region of ssDNA for miRNA 302d generation
acactcaaacatggaagcactta

SEQ ID NO: 16 - DNA, 23 bp, Artificial Sequence
  region of ssDNA for miRNA 369 generation
gaaaagatcaaccatgtattatt

SEQ ID NO: 17 - DNA, 23 bp, Artificial Sequence
  region of ssDNA for miRNA 302c generation
ccactgaaacatggaagcactta